(19) United States
(12) Patent Application Publication
(10) Pub. No.: US 2025/0331787 A1
(43) Pub. Date: Oct. 30, 2025

(54) LIFTING PLATFORM AND MEDICAL DEVICE

(71) Applicant: GE Precision Healthcare LLC, Waukesha, WI (US)

(72) Inventors: Qian Xu, Beijing (CN); Yigang Xia, Beijing (CN); Yifei Dong, Beijing (CN)

(21) Appl. No.: 19/195,274

(22) Filed: Apr. 30, 2025

(30) Foreign Application Priority Data
Apr. 30, 2024 (CN) 202420929696.5

(51) Int. Cl. A61B 6/04; A61B 6/03

(52) U.S. Cl. CPC A61B 6/0407; A61B 6/032

(57) ABSTRACT

A lifting platform of a medical device includes a base; a support platform, disposed on an upper side of the base; a support assembly, connecting the support platform and the base; a drive assembly, connected to the support assembly and driving the support assembly to move for lifting and lowering the support platform; a locking assembly, connected to the support assembly and used to lock movement of the support assembly; and a locking state switching assembly, detachably connected to the locking assembly to switch the locking assembly between a locked state and an unlocked state, wherein when the locking state switching assembly is connected to the locking assembly, the locking assembly is in an unlocked state, and the support assembly is capable of moving freely; and when the locking state switching assembly is separated from the locking assembly, the locking assembly is in a locked state, and movement is prevented.

### CROSS-REFERENCE TO RELATED APPLICATIONS

This application claims priority to Chinese Application No. 202420929696.5, filed on Apr. 30, 2024, now Chinese Patent No, ZL202420929696.5, the disclosure of which is incorporated herein by reference in its entirety.

### TECHNICAL FIELD

Embodiments of the present application relate to the technical field of medical devices, and in particular, to a lifting platform and a medical device.

### BACKGROUND

Lifting platforms are widely used in medical devices. Taking a medical device being a medical imaging system as an example, radiation from a ray source is emitted toward an examined subject, and the examined subject is generally a patient in a medical diagnosis application. A portion of radiation passes through the examined subject and is received by a detector, to generate an output signal based on the amount or intensity of radiation received by the detector. Related processing is performed on the output information to generate a medical image that can be displayed for examination.

For a lying-position scanning mode, generally, the examined subject needs to lie on the lifting platform in a lying position. The lifting platform includes a base and a support platform disposed on an upper side of the base. A support assembly is disposed between the base and the support platform, and the support assembly drives the support platform to move, so as to move the examined subject on the support platform to a desired position.

### SUMMARY

The inventor has found that: in order to prevent a support platform from being undesirably displaced or lifted when a user performs debugging or repair on a lifting platform, some locking apparatuses generally need to be mounted in the lifting platform to support or fasten the support platform. After debugging or repair ends, the user removes these locking apparatuses from the lifting platform to unlock the support platform, so that the support platform can be lifted freely under the drive of the support assembly. In the above design, the user needs to mount additional locking apparatuses on the lifting platform to support or fasten the support platform, and if a locking apparatus is lost or no proper locking apparatus can be found, locking cannot be performed reliably. Alternatively, if the user forgets to mount the locking apparatus, the support platform may be suddenly lowered or the like. This may easily lead to a safety problem or even serious injury. In addition, in the above design, the user can unlock the support platform only by removing the locking apparatus from the lifting platform. This easily leads to a safety problem in case of misoperation.

In view of at least one of the above technical problems, provided in embodiments of the present application are a lifting platform and a medical device.

According to an aspect of the embodiments of the present application, a lifting platform is provided. The lifting platform includes a base; a support platform, disposed on an upper side of the base; a support assembly, connecting the support platform and the base; a drive assembly, connected to the support assembly and driving the support assembly to move for lifting and lowering the support platform; a locking assembly, connected to the support assembly and used to lock movement of the support assembly; and a locking state switching assembly, detachably connected to the locking assembly to switch the locking assembly between a locked state and an unlocked state, wherein when the locking state switching assembly is connected to the locking assembly, the locking assembly is in an unlocked state, and the support assembly is capable of moving freely; and when the locking state switching assembly is separated from the locking assembly, the locking assembly is in a locked state, and movement of the support assembly is prevented.

According to another aspect of the embodiments of the present application, the locking state switching assembly covers a component to be repaired of the support assembly or the drive assembly.

According to another aspect of the embodiments of the present application, the component to be repaired comprises a connection portion between the support assembly and the drive assembly.

According to another aspect of the embodiments of the present application, the support assembly comprises a first support portion and a second support portion that are disposed crosswise, and the locking assembly comprises: a blocking member, extending to a movement path of the first support portion or the second support portion when the locking assembly is in a locked state.

According to another aspect of the embodiments of the present application, the first support portion comprises two arm portions that are opposite to each other, the second support portion is located between the two arm portions, and the blocking member is disposed on an arm portion of the first support portion.

According to another aspect of the embodiments of the present application, there are a plurality of blocking members, and the plurality of blocking members are separately disposed on the two arm portions of the first support portion.

According to another aspect of the embodiments of the present application, the locking assembly further includes a housing, wherein the housing comprises a first housing and a second housing, the first housing is fixedly connected to the first support portion, and the blocking member is movably mounted on the first housing; and the second housing is connected to a first end of the blocking member that is away from the support assembly, and the blocking member drives the second housing to move in a direction away from or toward the first support portion.

According to another aspect of the embodiments of the present application, when the locking state switching assembly is connected to the locking assembly, a portion of the locking state switching assembly is located between the second housing and the first support portion, or a portion of the locking state switching assembly is located between the second housing and the first housing.

According to another aspect of the embodiments of the present application, when the locking state switching assembly is separated from the locking assembly, the second housing abuts against the first support portion, and the first housing is accommodated within the second housing, or the second housing abuts against the first housing.

According to another aspect of the embodiments of the present application, the locking assembly further includes a first housing, wherein the first housing is fixedly connected to the first support portion, the blocking member is movably mounted on the first housing, a first end of the blocking member that is away from the support assembly comprises an abutting portion, and when the locking state switching assembly is connected to the locking assembly, a portion of the locking state switching assembly is located between the abutting portion and the first support portion, or a portion of the locking state switching assembly is located between the abutting portion and the first housing; or when the locking state switching assembly is separated from the locking assembly, the abutting portion abuts against the first housing.

According to another aspect of the embodiments of the present application, the locking assembly further includes an elastic component, located between the first housing and the blocking member to apply a force to the blocking member to move the blocking member toward the second support portion.

According to another aspect of the embodiments of the present application, the elastic component is disposed on an outer peripheral side or an inner side of the blocking member.

According to another aspect of the embodiments of the present application, the elastic component is accommodated within the first housing, one end of the elastic component abuts against the first housing, and the other end thereof abuts against the blocking member.

According to another aspect of the embodiments of the present application, a medical device is provided. The medical device comprises the above-described lifting platform.

One of the beneficial effects of the embodiments of the present application is that a locking assembly, which can prevent movement of the support assembly of the lifting platform, and a locking state switching assembly, which is detachably connected to the locking assembly, are disposed in the lifting platform. In this way, when the locking state switching assembly is connected to the locking assembly, the locking assembly is in an unlocked state, and the support assembly is capable of moving freely. When the locking state switching assembly is separated from the locking assembly, the locking assembly is in a locked state, and movement of the support assembly is prevented. That is, the locking state switching assembly is separated from the locking assembly, so that the locking assembly can be switched from an unlocked state to a locked state to lock the lifting platform. This can simplify a locking operation and help prevent a safety problem. In addition, the locking state switching assembly is connected to the locking assembly, so that the locking assembly can be switched from a locked state to an unlocked state to unlock the lifting platform. This can help prevent the lifting platform from being unlocked due to misoperation, and help prevent a safety problem.

With reference to the following description and drawings, specific implementations of the embodiments of the present application are disclosed in detail, and the way in which the principles of the embodiments of the present application can be employed are illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT system for CT imaging according to an embodiment of the present application;

FIG. 2 is another schematic diagram of a CT system for CT imaging according to an embodiment of the present application;

FIG. 3 is a schematic diagram of a lifting platform according to an embodiment of the present application;

FIG. 4 is a schematic diagram of a locking state switching assembly according to an embodiment of the present application;

FIG. 5 is a schematic diagram of a connection portion according to an embodiment of the present application;

FIG. 6 is a schematic diagram of a support assembly, a locking assembly, and a locking state switching assembly according to an embodiment of the present application;

FIG. 7 and FIG. 8 are schematic diagrams of a partial structure of a locking assembly and a first support portion according to an embodiment of the present application; and

FIG. 9 and FIG. 10 are schematic diagrams of a partial structure of a locking assembly, a first support portion, and a second support portion according to an embodiment of the present application.

### DETAILED DESCRIPTION

Specific embodiments of the present application are described below. It should be noted that in the specific description of said embodiments, for the sake of brevity and conciseness, the present description cannot describe all of the features of the actual embodiments in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. In addition, it should also be understood that although efforts made in such development processes may be complex and tedious, for those of ordinary skill in the art related to the content disclosed in the present application, some design, manufacture, or production changes made on the basis of the technical content disclosed in the present application are only common technical means, and should not be construed as the content of the present application being insufficient.

Unless defined otherwise, technical terms or scientific terms used in the claims and description should have the usual meanings that are understood by those of ordinary skill in the technical field to which the present application belongs. Terms such as “first”, “second”, and similar terms used in the description and claims of the present patent application do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or subject preceding the terms “include” or “comprise” encompasses elements or subjects and equivalent elements thereof listed after the terms “include” or “comprise”, and do not exclude other elements or subjects. The terms “connect” or “link” and similar words are not limited to physical or mechanical connections, and are not limited to direct or indirect connections.

In embodiments of the present application, a lifting platform may be applied to various devices. The device includes, for example, a medical device or an industrial device. Taking the medical device as an example, the medical device includes, for example, devices related to various medical imaging scenarios. The medical imaging scenario includes but is not limited to magnetic resonance imaging (MRI), computed tomography (CT), ultrasound imaging, X-ray imaging, positron emission computed tomography (PET), single photon emission computed tomography (SPECT), PET/CT, PET/MR, or any other suitable medical imaging scenario.

In the embodiments of the present disclosure, the CT scenario is taken as an example to describe the lifting platform and the medical device in the present application. Those skilled in the art will understand that the present application is also applicable to other radiographic imaging systems or imaging systems based on other high-frequency electromagnetic energy.

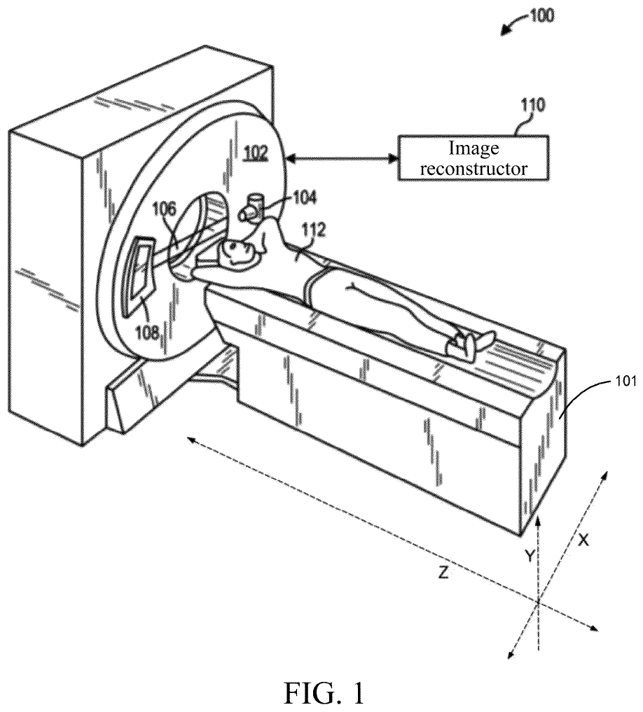

FIG. 1 is a schematic diagram of a CT system for CT imaging according to an embodiment of the present application. As shown in FIG. 1, a CT system 100 is configured to image a scanned subject 112 (for example, a patient, an inanimate subject, and an implant, a stent, and/or a contrast agent present in a body).

The CT system 100 includes a lifting platform 101, the scanned subject 112 lies on the lifting platform 101, and the lifting platform 101 drives the subject 112 to be displaced, for example, displaced along at least one of the coordinate axes X, Y, Z in FIG. 1.

The CT system 100 includes a gantry 102. An X-ray source 104 is mounted on the gantry 102, and the X-ray source 104 is configured to emit an X-ray beam 106 for imaging the scanned subject 112. Specifically, the X-ray source 104 is configured to project the X-ray beam 106 toward a detector assembly 108 positioned on an opposite side of the gantry 102.

In some embodiments, the X-ray source 104 emits a fan-shaped or cone-shaped ray beam. The ray beam is collimated to be located on an X-Y-Z plane, namely, an imaging plane, of a Cartesian coordinate system. The X-ray beam passes through the scanned subject 112 and is attenuated by the scanned subject 112 before being projected on the detector assembly 108. Therefore, the ray intensity received on the detector assembly 108 depends on the degree of attenuation on the ray by the scanned subject 112. The detector assembly 108 may include a plurality of detector units forming an array, and each detector unit detects a signal representing the intensity of a corresponding ray beam. The signal detected by each detector unit may be provided as distributed projection data (or image data) for subsequent processing.

In some embodiments, the CT system 100 further includes an image reconstructor 110, configured to receive the projection data and perform image reconstruction, to obtain a volumetric image of the scanned subject 112. The image may specifically include a three-dimensional volumetric image or an image corresponding to one or a plurality of two-dimensional slices of the scanned subject 112. The reconstructed image may be transmitted to a processing device and stored in a mass memory coupled to the processing device.

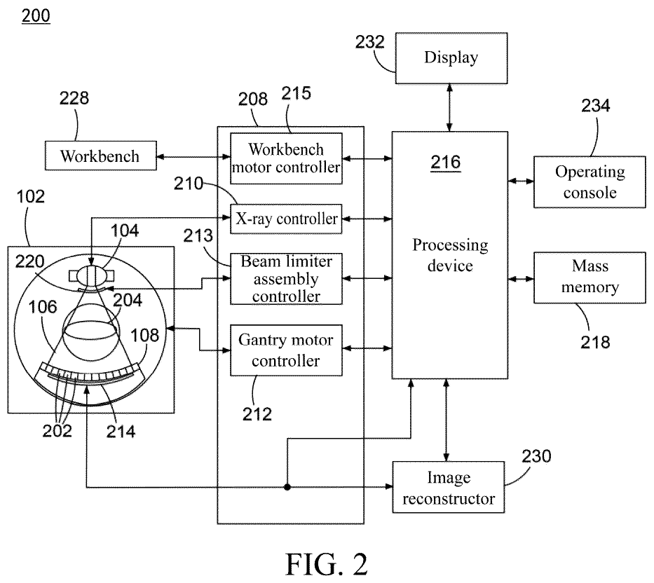

FIG. 2 is another schematic diagram of a CT system for CT imaging according to an embodiment of the present application. The CT system 200 shown in FIG. 2 is similar to the CT system 100 shown in FIG. 1. As shown in FIG. 2, the CT system 200 is configured to image a scanned subject 204 (for example, the scanned subject 112 in FIG. 1).

The CT system 200 includes a detector assembly 108, the detector assembly includes a plurality of detector units 202, and the plurality of detector units 202 together detect an X-ray beam 106 passing through the scanned subject 204 to acquire corresponding projection data. In an example, the detector assembly includes a plurality of rows of detector units 202, which may be configured to acquire projection data corresponding to a plurality of slices. The detector unit 202 may be an energy integrating detector, a photon counting detector, an energy discriminating detector, or any other suitable radiation detector. The detector unit 202 may use a scintillator and generate an analog signal, or the detector unit 202 may be a direct conversion detector (that is, a detector that does not use a scintillator), such as a detector based on a silicon strip or another semiconductor material. When the semiconductor sensor itself is exposed to an X-ray, the detector generates a measurable signal.

In some embodiments, the CT system 200 further includes a beam limiter assembly 220, the beam limiter assembly 220 may be positioned in a propagation direction of an X-ray source 104 and adjacent to the X-ray source, and the beam limiter assembly 220 may limit the shape and/or extent of a high-intensity region of the X-ray beam 106 to limit X-ray exposure to an area outside a region of interest of the scanned subject 204. Specifically, the beam limiter assembly 220 may be mounted between the X-ray source 104 and the scanned subject 204 and positioned near the X-ray source 104. The beam limiter assembly 220 may include one or a plurality of blades (or baffles). The one or the plurality of blades forms/form an opening. The one or the plurality of blades blocks/block a portion of the X-ray, and the opening is configured to allow another portion of the ray to pass through. In this way, the shape and/or extent of the projected X-ray beam are/is limited.

In some embodiments, the CT system 200 may further include a collimator (not shown in the figure). The collimator may be disposed between the scanned subject 204 and the detector assembly 208 and adjacent to the detector assembly 208. The collimator is generally made of a material that blocks or absorbs the X-ray. Therefore, the X-ray that is reflected or deflected via the scanned subject 204 can be blocked by the collimator to prevent the X-ray from being projected to the detector assembly 208. Conversely, the X-ray 106 traveling in a relatively straight path from the X-ray source 104 to the detector assembly 208 is not obstructed by the collimator 222 and can be projected to the detector assembly 208.

In some embodiments, the CT system 200 also includes a control mechanism 208, and the control mechanism 208 may include an X-ray controller 210 and a gantry motor controller 212. During scanning to acquire the projection data, the X-ray controller 210 is configured to control an operation of the X-ray source 104 (for example, provide power and timing signals to the X-ray source 104 and determine/track a focal point/focal spot position of the X-ray source), and the gantry motor controller 212 is configured to control, based on an imaging requirement, the gantry 102 and a component mounted thereon to rotate around the scanned subject 112 at an appropriate speed, so that an angle/position at which the X-ray beam 106 intersects the scanned subject 112 continuously changes. The projection data acquired by the detector assembly 108 at each angle is referred to as a view, and scanning performed on the scanned subject 112 includes a set of views taken at different angles during one time of rotation of the X-ray source 104 and the detector assembly 108 around the scanned subject 112.

In some embodiments, the control mechanism 208 further includes a lifting platform motor controller 215, and the lifting platform motor controller 215 may further control a lifting platform 228 (for example, the scanning lifting platform 101 in FIG. 1). Specifically, the lifting platform motor controller 215 may move the lifting platform 228, so that the scanned subject 204 on the lifting platform 228 is moved totally or partially to pass through the opening of the gantry 102 in FIG. 1, so as to position the scanned subject 204 within a scanning region limited by the gantry 102 to acquire projection data corresponding to the scanned subject 204.

In some embodiments, the control mechanism 208 further includes a beam limiter assembly controller 213 configured to control an operation of the beam limiter assembly 220, for example, may control movement of the one or the plurality of blades of the beam limiter assembly 220 (or a beam limiter assembly 300 as will be described below) to adjust the position or the width of the opening. In some embodiments, such adjustment may be performed based on a result of tracking the focal point of the X-ray source by the X-ray controller 210.

A detector array composed of the detector units 202 includes a full-shadow region for receiving full-shadow data and a penumbra region for receiving penumbra data. When the X-ray source 104 emits the X-ray beam 106 at different focal spot positions, the full-shadow region can receive the X-ray beam at all focal spot positions, whereas the penumbra region may only receive the X-ray beam emitted at a part of focal spot positions. In some embodiments, the full-shadow region is generally formed as a regular array (for example, rectangular) to adapt to the shape of the X-ray beam limited via, for example, the beam limiter assembly 220 having a square opening. In order to track the focal spot position, it is necessary to dispose a small quantity of additional detectors at an edge portion outside the regular array to form the above penumbra region, so that an irregular shape is formed as a whole, leading to high design costs.

In some embodiments, the CT system 200 further includes a data acquisition system (DAS) 214, configured to sample and digitize data received by the detector units 202 to convert the data into projection data. As further described herein, the data sampled and digitized by the DAS 214 is transmitted to a processing device 216.

The processing device 216 may include a computer device. In an example, the processing device 216 stores the data into a storage device or a mass memory 218, or the data sampled and digitized by the DAS 214 is directly transmitted to a mass memory 218. For example, the storage device 218 may include a hard disk drive, a floppy disk drive, a compact disc-read/write (CD-R/W) drive, a digital versatile disc (DVD) drive, a flash drive, a solid-state storage drive, and/or the like. An associated display 232 allows the operator to observe the reconstructed image and other data from the processing device. The display 232 may also allow the operator to select a region of interest and/or request patient information via a graphical user interface for subsequent processing.

In some embodiments, the CT system 200 further includes an operating console 234 that is operatively coupled to the processing device 216, and the processing device 216 may receive instructions and parameters that are input by an operator via the operating console 234. The operating console has an operator interface in a specific form, such as a keyboard, a mouse, a voice activated controller, or any other suitable input apparatus.

Additionally, the processing device 216 is configured to send commands or parameters to one or a plurality of the DAS 214, the X-ray controller 210, the gantry motor controller 212, the beam limiter assembly controller 213, or the lifting platform motor controller 215 to control a system operation, such as data acquisition and/or processing. The processing device 216 may control a system operation based on operator input.

In some embodiments, the processing device 216 may calculate, based on the penumbra data and the full-shadow data in the data received from the DAS, a parameter (for example, a Z-ratio) for tracking the focal point of the X-ray source 104, and send the parameter to the X-ray controller 210. The X-ray controller 210 tracks the focal point position of the X-ray source 104 based on the received parameter (for example, the Z-ratio). The processing device 216 may further receive, from the X-ray controller 210, a result of tracking the focal point, and send the result to the beam limiter assembly controller 213, so that an operation of the beam limiter assembly 220 (or the beam limiter assembly 300) can be controlled based on a current focal point position.

In some embodiments, the CT system 200 includes an image reconstructor 230, the image reconstructor 230 is configured to receive projection data from the DAS 214 and perform image reconstruction, and a reconstructed image may be transmitted to the processing device 216 to generate patient information for diagnosis and evaluation. The processing device 216 is configured to store the reconstructed image and the patient information in the mass memory 218. In some embodiments, the processing device 216 may transmit the reconstructed image to the display 232, and the display 232 may be communicatively coupled to the processing device 216 and/or the image reconstructor 230.

Only one example of the CT system suitable for applying the embodiments of the present application is described above. Those skilled in the art will recognize that the embodiments of the present application are also applicable to any imaging system or device based on other radioactive rays or high-frequency electromagnetic energy.

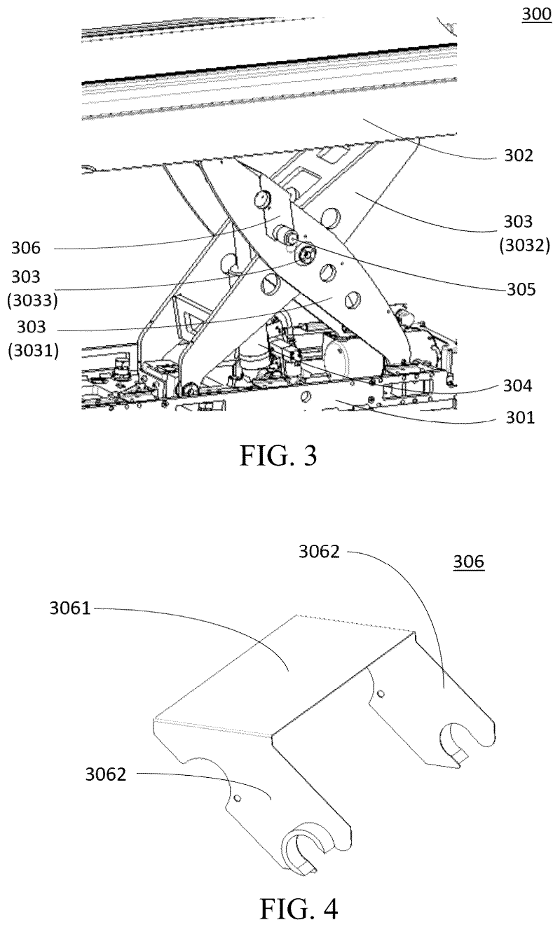

FIG. 3 is a schematic diagram of a lifting platform according to an embodiment of the present application. As shown in FIG. 3, a lifting platform 300 includes a base 301, a support platform 302, a support assembly 303, a drive assembly 304, a locking assembly 305, and a locking state switching assembly 306.

As shown in FIG. 3, the support platform 302 is disposed on an upper side of the base 301. The support assembly 303 connects the support platform 302 and the base 301. The drive assembly 304 is connected to the support assembly 303, and drives the support assembly 303 to move for lifting and lowering the support platform 302.

The locking assembly 305 is connected to the support assembly 303 to lock movement of the support assembly 303. The locking state switching assembly 306 is detachably connected to the locking assembly 305 to switch the locking assembly 305 between a locked state and an unlocked state.

When the locking state switching assembly 306 is connected to the locking assembly 305, the locking assembly 305 is in an unlocked state, and the support assembly 303 is capable of moving freely. When the locking state switching assembly 306 is separated from the locking assembly 305, the locking assembly 305 is in a locked state, and movement of the support assembly 303 is prevented.

Based on the above embodiment, the locking assembly 305 that can prevent movement of the support assembly 303 of the lifting platform 300 and the locking state switching assembly 306 detachably connected to the locking assembly 303 are disposed in the lifting platform 300. In this way, when the locking state switching assembly 306 is connected to the locking assembly 305, the locking assembly 305 is in an unlocked state, and the support assembly 303 is capable of moving freely. When the locking state switching assembly 306 is separated from the locking assembly 305, the locking assembly 305 is in a locked state, and movement of the support assembly 303 is prevented. That is, the locking state switching assembly 306 is separated from the locking assembly 305, so that the locking assembly 305 can be switched from an unlocked state to a locked state to lock the lifting platform 300. This can simplify a locking operation and help prevent a safety problem. In addition, the locking state switching assembly 306 is connected to the locking assembly 305, so that the locking assembly 305 can be switched from a locked state to an unlocked state to unlock the lifting platform 300. This can help prevent the lifting platform 300 from being unlocked due to misoperation, and help prevent a safety problem.

In some embodiments, as shown in FIG. 3, when the locking state switching assembly 306 is connected to the locking assembly 305, the locking state switching assembly 306 may cover a component to be repaired of the support assembly 303 or the drive assembly 304. For example, the locking state switching assembly 306 may cover at least a portion of the component to be repaired.

Based on the above structure, if a user wants to repair the component to be repaired, the user needs to first release coverage of the component to be repaired by the locking state switching assembly 306. To release the coverage, the user needs to first separate the locking state switching assembly 306 from the locking assembly 305. As described above, when the locking state switching assembly 306 is separated from the locking assembly 305, the locking assembly 305 is switched from an unlocked state to a locked state, and movement of the support assembly 303 is prevented, so that the support platform 302 can be prevented from being undesirably displaced or lowered.

Therefore, with the above structure, the lifting platform 300 can be reliably locked before the component to be repaired is repaired, thereby implementing a poka-yoke function. This can provide reliable safety protection, and effectively prevent a safety problem. Further, with the locking state switching assembly 306, the component to be repaired can be covered, and the locking assembly 305 can be switched to a locked state. Therefore, a poka-yoke function can be implemented by using a small quantity of components. This helps simplify the structure of the lifting platform 300.

In some embodiments, the locking state switching assembly 306 may be of a variety of structures that can cover or shield the component to be repaired. FIG. 4 is a schematic diagram of a locking state switching assembly according to an embodiment of the present application. As shown in FIG. 4, the locking state switching assembly 306 may be a housing, so that the component to be repaired can be reliably covered or shielded.

As shown in FIG. 4, the locking state switching assembly 306 includes a shielding portion 3061 and claw portions 3062. The shielding portion 3061 is configured to cover or shield the component to be repaired, and the claw portions 3062 are configured to be connected to the locking assembly 305. A specific connection mode is described later.

In some embodiments, the component to be repaired of the lifting platform 300 may include a connection portion between the support assembly 303 and the drive assembly 304. Therefore, before the drive assembly 304 is repaired or replaced, it can be ensured that the lifting platform 300 is in a locked state, so that the support platform 302 is not abruptly lowered due to repair or removal of the drive assembly 304. The present application is not limited thereto. The component to be repaired may be another component of the lifting platform 300.

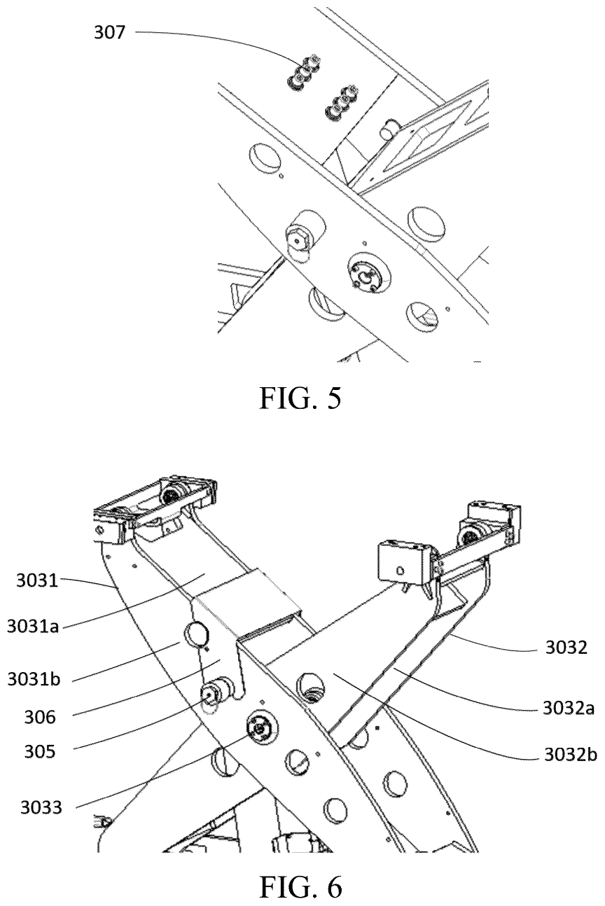

In some embodiments, the connection portion for connecting the drive assembly 304 and the support assembly 303 may be in various forms. FIG. 5 is a schematic diagram of a connection portion according to an embodiment of the present application. As shown in FIG. 5, a connection portion 307 may include at least one screw. The at least one screw fastens one end of the drive assembly 304 to the support assembly 303. A structure of the support assembly is described below by using an example with reference to the accompanying drawings.

As shown in FIG. 3, the support assembly 303 may include a first support portion 3031 and a second support portion 3032 that are disposed crosswise. For example, the support assembly 303 is in the form of a scissor, two scissor arms (namely, the first support portion 3031 and the second support portion 3032) are connected by using a shaft 3033, and the two scissor arms may rotate around the shaft 3033.

Upper ends of the first support portion 3031 and the second support portion 3032 are connected to the support platform 302, and lower ends of the first support portion 3031 and the second support portion 3032 are connected to the base 301.

FIG. 6 is a schematic diagram of a support assembly, a locking assembly, and a locking state switching assembly according to an embodiment of the present application. As shown in FIG. 6, in the support assembly 303, the first support portion 3031 includes two arm portions 3031a and 3031b that are opposite to each other. The second support portion 3032 may be located between the two arm portions 3031a and 3031b. That is, the two arm portions 3031a and 3031b sandwich the second support portion 3032. The two arm portions 3031a and 3031b of the first support portion 3031 are fixedly connected to each other and synchronously rotate around the shaft 3033.

In some embodiments, as shown in FIG. 6, the second support portion 3032 may also include two arm portions 3032a and 3032b. The two arm portions 3032a and 3032b of the second support portion 3032 are disposed between the two arm portions 3031a and 3031b of the first support portion 3031. The two arm portions 3032a and 3032b of the second support portion 3032 are fixedly connected to each other and synchronously rotate around the shaft 3033. Therefore, strength of the support assembly 303 can be ensured, and the support platform 302 can be stably supported.

For convenience of description, in the following descriptions, space between the two arm portions 3031a and 3031b of the first support portion 3031 is referred to as an inner side of the first support portion 3031. Accordingly, a side of the arm portion 3031a that is away from the arm portion 3031b and a side of the arm portion 3031b that is away from the arm portion 3031a are referred to as an outer side of the first support portion 3031.

Similarly, space between the two arm portions 3032a and 3032b of the second support portion 3032 is referred to as an inner side of the second support portion 3032. Accordingly, a side of the arm portion 3032a that is away from the arm portion 3032b and a side of the arm portion 3032b that is away from the arm portion 3032a are referred to as an outer side of the second support portion 3032.

The above is only an exemplary description of the support assembly 303. The support assembly 303 may be of another structure. This is not specifically limited in the present application. A mode in which the drive assembly drives the support assembly is described below by using an example with reference to the accompanying drawings. In some embodiments, the drive assembly 304 is, for example, a cylinder, a hydraulic cylinder, a linear actuator, a ball screw, or the like.

As shown in FIG. 3, a lower end of the drive assembly 304 is connected to the base 301. As shown in FIG. 5, an upper end of the drive assembly 304 is connected to at least one of the first support portion 3031 and the second support portion 3032 by using the connection portion 307. The heights of the upper end of the first support portion 3031 and the upper end of the first support portion 3032 are adjusted by adjusting a tilt angle of the first support portion 3031 or the second support portion 3032, so as to adjust the height of the support platform 302.

The above is only an exemplary description of the mode in which the drive assembly drives the support assembly. The drive assembly 304 may drive the support assembly 303 in another mode. This is not specifically limited in the present application. The structure of the locking assembly is described below by using an example with reference to the accompanying drawings.

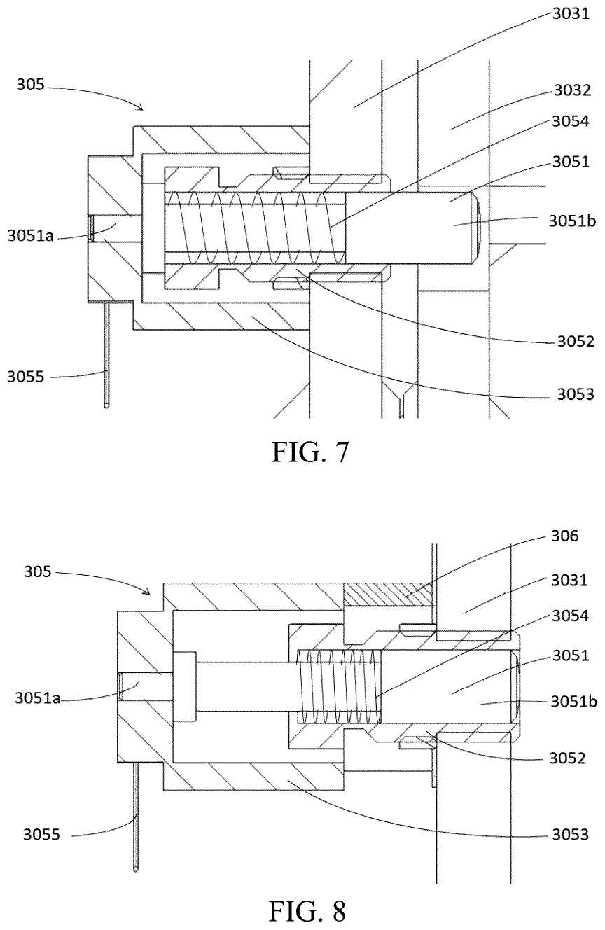

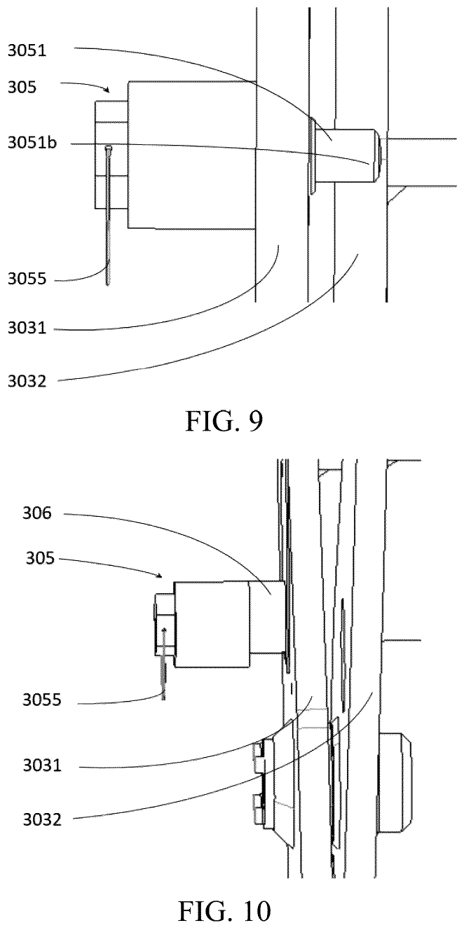

FIG. 7 and FIG. 8 are schematic diagrams of a partial structure of a locking assembly and a first support portion according to an embodiment of the present application. FIG. 9 and FIG. 10 are schematic diagrams of a partial structure of a locking assembly, a first support portion, and a second support portion according to an embodiment of the present application. FIG. 7 and FIG. 9 correspond to a locked state of the locking assembly 305, and FIG. 8 and FIG. 10 correspond to an unlocked state of the locking assembly 305. In some embodiments, as shown in FIG. 7 and FIG. 9, the locking assembly 305 includes a blocking member 3051. When the locking assembly 305 is in a locked state, the blocking member 3051 extends to a movement path of the first support portion 3031 or the second support portion 3032. Therefore, movement of the first support portion 3031 or the second support portion 3032 along the movement path can be blocked, so that the lifting platform 300 can be locked.

In some embodiments, as shown in FIG. 8 and FIG. 10, when the locking assembly 305 is in an unlocked state, the blocking member 3051 (shown in FIG. 8) is located outside the movement path of the first support portion 3031 and the second support portion 3032. Therefore, the first support portion 3031 and the second support portion 3032 can move along the movement path, so that the lifting platform 300 can be unlocked.

In some embodiments, as shown in FIG. 7 to FIG. 10, the blocking member 3051 may be disposed on the arm portion of the first support portion 3031 of the support assembly 303. The blocking member 3051 is disposed on the first support portion 3031 on the outer side, so that the user can connect or separate the locking state switching assembly 306 and the locking assembly 305. The present application is not limited thereto. The blocking member 3051 may be disposed on the arm portion of the second support portion 3032.

In some embodiments, as shown in FIG. 9, when the locking assembly 305 is in a locked state, a second end 3051b of the blocking member 3051 may be located at a position at which the second end protrudes from the first support portion 3031 toward the inner side, so as to be located on the movement path of the second support portion 3032.

As shown in FIG. 10, when the locking assembly 305 is in an unlocked state, a second end 3051b (not shown in the figure) of the blocking member 3051 may be located at a position at which the second end does not protrude from the first support portion 3031 toward the inner side, for example, the blocking member 3051 may be located at a position at which the second end 3051b is flush with an inner side surface of the first support portion 3031. Therefore, movement of the second support portion 3032 can be prevented from being blocked.

In some embodiments, there may be a plurality of blocking members 3051, and the plurality of blocking members 3051 may be separately disposed on the two arm portions of the first support portion 3031. Therefore, the plurality of blocking members 3051 can prevent the first arm 3031a of the first support portion 3031 and the first arm 3032a of the second support portion 3032 from rotating around the shaft 3033, and can also prevent the second arm 3031b of the first support portion 3031 and the second arm 3032b of the second support portion 3032 from rotating around the shaft 3033. This can more reliably prevent the support assembly 303 from moving.

In some embodiments, the locking assembly 305 may further include another component. For example, as shown in FIG. 7 and FIG. 8, the locking assembly 305 may further include a housing. The housing may include a first housing 3052 and a second housing 3053. The first housing 3052 is fixedly connected to the first support portion 3031, and the blocking member 3051 is movably mounted on the first housing 3052. The second housing 3053 is connected to a first end 3051a of the blocking member 3051 that is away from the support assembly 303, and the blocking member 3051 can drive the second housing 3053 to move in a direction (for example, a horizontal direction) away from or toward the first support portion 3031.

In some embodiments, the first housing 3052 may be fixedly connected to the first support portion 3031 in various modes. For example, a through hole is disposed at a predetermined position of the first support portion 3031, one end of the first housing 3052 may be disposed within the through hole, and one end of the first housing 3052 is fastened within the through hole of the first support portion 3031 by using a nut disposed on an outer peripheral side of the first housing 3052. For another example, a matching thread may be disposed on an inner wall of the through hole and the outer periphery of the first housing 3052, and one end of the first housing 3052 is fastened within the through hole via threaded connection. For another example, the first support portion 3031 may be fastened within the via hole through bonding, welding, riveting, or the like.

In some embodiments, the second housing 3052 may be connected to the first end 3051a of the blocking member 3051 in various modes. For example, the second housing 3052 may be connected to the first end 3051a of the blocking member 3051 in a mode similar to the above mode in which the first housing 3052 is connected to the first support portion 3031.

In some embodiments, as shown in FIG. 7 and FIG. 8, when one end of the first housing 3052 is fixedly connected to the first support portion 3031, the other end of the first housing 3052 protrudes from the first support portion 3031 toward the outer side. The second housing 3053 is also disposed on the outer side of the first support portion 3031. Therefore, even when an interval between the first support portion 3031 and the second support portion 3032 is small, the blocking member 3051 can be disposed on the support assembly 303. In addition, it is convenient to separate or connect the locking state switching assembly 306 and the locking assembly 305.

In some embodiments, as shown in FIG. 7, when the locking state switching assembly 306 is separated from the locking assembly 305, the second housing 3053 abuts against the first support portion 3031, and the first housing 3052 is accommodated within the second housing 3053. In a locked state, the blocking member 3051 blocks movement of the second support member 3032, and a large force is applied to the blocking member 3051. The second housing 3053 abuts against the first support portion 3031, so that the locking assembly 305 can be prevented from being displaced, and the second support member 3032 can be reliably prevented from moving. In addition, when the locking assembly 305 is in a locked state, the first housing 3052 is accommodated within the second housing 3053, so that the first housing 3052 can be prevented from being undesirably displaced due to an external force, thereby further improving locking reliability.

In some embodiments, as shown in FIG. 8, when the locking state switching assembly 306 is connected to the locking assembly 305, a portion of the locking state switching assembly 306 is located between the second housing 3053 and the first support portion 3031. The blocking member 3051 is driven by the second housing 3053, and the second end 3051b of the blocking member 3051 is accommodated within the first housing 3052.

In some embodiments, a portion (for example, the above claw portions 3062) of the locking state switching assembly 306 is engaged with the second housing 3053 and the first support portion 3031. The width of this portion may be equal to a length by which the second end 3051b of the blocking member 3051 protrudes from the first support portion 3031 toward the inner side in a locked state. The width of this portion is set to an appropriate value, so that the second end 3051b of the blocking member 3051 can be located at a position at which movement of the second support portion 3032 is blocked. The present application is not limited thereto. The width of this portion may be greater than the length by which the second end 3051b of the blocking member 3051 protrudes from the first support portion 3031 toward the inner side in a locked state.

The present application is not limited thereto. In some embodiments, the second housing 3053 may abut against the first housing 3052 when the locking state switching assembly 306 is separated from the locking assembly 305. For example, second housing 3053 abuts against the first housing 3052 in the horizontal direction. Alternatively, in some embodiments, when the locking state switching assembly 306 is connected to the locking assembly 305, a portion of the locking state switching assembly 306 may be located between the second housing 3053 and the first housing 3052.

In some embodiments, in order to enable the blocking member 3051 to reliably protrude from the first support portion 3031 toward the second support portion 3032, an elastic component may be disposed in the locking assembly 305. The elastic component may be located between the first housing 3052 and the blocking member 3051 to apply a force to the blocking member 3051 to move the blocking member 3051 toward the second support portion 3032.

As shown in FIG. 7 and FIG. 8, the elastic component 3054 may be disposed on an outer peripheral side of the blocking member 3051. For example, the elastic component 3054 is accommodated within the first housing 3052, one end of the elastic component 3054 abuts against the first housing 3052, and the other end thereof abuts against the blocking member 3051.

Therefore, when the locking state switching assembly 306 is connected to the locking assembly 305, the second end 3051b of the blocking member 3051 is accommodated within the first housing 3052, and the elastic component 3054 is in a first compressed state. After the locking state switching assembly 306 is separated from the locking assembly 305, under the action of the elastic component 3054, the second end 3051b of the blocking member 3051 protrudes from the first housing 3052, and the elastic component 3054 is in a natural state or a second compressed state. In the second compressed state, a compression degree is lower than that in the first compressed state. The present application is not limited thereto. The elastic component 3054 may be disposed on the inner side of the blocking member 3051.

In some embodiments, as shown in FIG. 7 to FIG. 10, the locking assembly 305 may further include a force application portion 3055. The force application portion 3055 is connected to the second housing 3053. The force application portion 3055 is disposed, so that the user can apply a force to the second housing 3053 in a direction away from the support assembly 303. Therefore, the locking state switching assembly 306 can be placed between the second housing 3053 and the first support portion 3031 or between the second housing 3053 and the first housing 3052.

The present application is not limited thereto. The locking assembly 305 may be of another structure. For example, the locking assembly 305 may not be provided with the above second housing 3053. For example, the first end 3051a of the blocking member 3051 that is away from the support assembly 303 may include an abutting portion. When the locking state switching assembly 306 is connected to the locking assembly 305, a portion of the locking state switching assembly 306 is located between the abutting portion and the first support portion 3031, or a portion of the locking state switching assembly 306 is located between the abutting portion 306 and the first housing 3052. When the locking state switching assembly 306 is separated from the locking assembly 305, the abutting portion may abut against the first housing 3052.

In some embodiments, the outer diameter of the abutting portion may be greater than the outer diameter of an intermediate portion between the first end 3051a and the second end 3051b of the blocking member 3051. The present application is not limited thereto. The abutting portion may be of another structure, provided that the abutting portion can abut against a portion of the locking state switching assembly 306.

The present application is not limited thereto. The locking assembly 305 may not include the elastic component. For example, when the locking state switching assembly 306 is separated from the locking assembly 305, the second housing 3053 may abut against the first support portion 3031 or the first housing 3052 via magnetic attraction.

It is worth noting that only the individual components or modules relevant to this embodiment have been described above, but the present application is not limited thereto. The lifting platform may further include another component or module. For specific content of the component or module, refer to the related technology.

Based on the above embodiment, the locking assembly that can lock movement of the support assembly of the lifting platform and the locking state switching assembly detachably connected to the locking assembly are disposed in the lifting platform. In this way, when the locking state switching assembly is connected to the locking assembly, the locking assembly is in an unlocked state, and the support assembly is capable of moving freely. When the locking state switching assembly is separated from the locking assembly, the locking assembly is in a locked state, and movement of the support assembly is prevented. That is, the locking state switching assembly is separated from the locking assembly, so that the locking assembly can be switched from an unlocked state to a locked state to lock the lifting platform. This can simplify a locking operation and help prevent a safety problem. In addition, the locking state switching assembly is connected to the locking assembly, so that the locking assembly can be switched from a locked state to an unlocked state to unlock the lifting platform. This can help prevent the lifting platform from being unlocked due to misoperation, and help prevent a safety problem.

An embodiment of the present application further provides a medical device. The medical device includes the lifting platform described above. As shown in FIG. 1, the medical device may be a CT system 100 or the like.

Based on the above embodiment, the locking assembly that can lock movement of the support assembly of the lifting platform and the locking state switching assembly detachably connected to the locking assembly are disposed in the lifting platform. In this way, when the locking state switching assembly is connected to the locking assembly, the locking assembly is in an unlocked state, and the support assembly is capable of moving freely. When the locking state switching assembly is separated from the locking assembly, the locking assembly is in a locked state, and movement of the support assembly is prevented. That is, the locking state switching assembly is separated from the locking assembly, so that the locking assembly can be switched from an unlocked state to a locked state to lock the lifting platform. This can simplify a locking operation and help prevent a safety problem. In addition, the locking state switching assembly is connected to the locking assembly, so that the locking assembly can be switched from a locked state to an unlocked state to unlock the lifting platform. This can help prevent the lifting platform from being unlocked due to misoperation, and help prevent a safety problem. The lifting platform is disposed in the medical device, so that a locking operation can be simplified during a process of operating the medical device, and it helps prevent a safety problem due to misoperation.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application. As used herein, the term “example” means serving as a non-limiting example, instance, or illustration. As used herein, the term “for example” introduces a list of one or a plurality of non-limiting examples, instances, or illustrations.

Preferred embodiments of the present application are described above with reference to the accompanying drawings. Many features and advantages of the embodiments are clear according to the detailed description. Therefore, the appended claims are intended to cover all these features and advantages that fall within the true spirit and scope of these embodiments. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the embodiments.

1. A lifting platform, characterized by comprising:
a base;
a support platform, disposed on an upper side of the base;
a support assembly, connecting the support platform and the base;
a drive assembly, connected to the support assembly and driving the support assembly to move for lifting and lowering the support platform;
a locking assembly, connected to the support assembly and used to lock movement of the support assembly; and
a locking state switching assembly, detachably connected to the locking assembly to switch the locking assembly between a locked state and an unlocked state, wherein
when the locking state switching assembly is connected to the locking assembly, the locking assembly is in an unlocked state, and the support assembly is capable of moving freely; and when the locking state switching assembly is separated from the locking assembly, the locking assembly is in a locked state, and movement of the support assembly is prevented.

2. The lifting platform according to claim 1, wherein
the locking state switching assembly covers a component to be repaired of the support assembly or the drive assembly.

3. The lifting platform according to claim 2, wherein
the component to be repaired comprises a connection portion between the support assembly and the drive assembly.

4. The lifting platform according to claim 1, wherein
the support assembly comprises a first support portion and a second support portion that are disposed crosswise; and
the locking assembly comprises:
a blocking member, extending to a movement path of the first support portion or the second support portion when the locking assembly is in a locked state.

5. The lifting platform according to claim 4, wherein
the first support portion comprises two arm portions that are opposite to each other, and the second support portion is located between the two arm portions; and
the blocking member is disposed on an arm portion of the first support portion.

6. The lifting platform according to claim 5, wherein
there are a plurality of blocking members, and the plurality of blocking members are separately disposed on the two arm portions of the first support portion.

7. The lifting platform according to claim 4, wherein the locking assembly further comprises:
a housing, the housing comprising a first housing and a second housing;
the first housing is fixedly connected to the first support portion, and the blocking member is movably mounted on the first housing; and
the second housing is connected to a first end of the blocking member that is away from the support assembly, and the blocking member drives the second housing to move in a direction away from or toward the first support portion.

8. The lifting platform according to claim 7, wherein
when the locking state switching assembly is connected to the locking assembly, a portion of the locking state switching assembly is located between the second housing and the first support portion, or a portion of the locking state switching assembly is located between the second housing and the first housing.

9. The lifting platform according to claim 7, wherein
when the locking state switching assembly is separated from the locking assembly, the second housing abuts against the first support portion, and the first housing is accommodated within the second housing, or the second housing abuts against the first housing.

10. The lifting platform according to claim 4, wherein the locking assembly further comprises:
a first housing, the first housing being fixedly connected to the first support portion, and the blocking member being movably mounted on the first housing;
a first end of the blocking member that is away from the support assembly comprises an abutting portion; and
when the locking state switching assembly is connected to the locking assembly, a portion of the locking state switching assembly is located between the abutting portion and the first support portion, or a portion of the locking state switching assembly is located between the abutting portion and the first housing;
or
when the locking state switching assembly is separated from the locking assembly, the abutting portion abuts against the first housing.

11. The lifting platform according to claim 7, wherein the locking assembly further comprises:
an elastic component, located between the first housing and the blocking member, applying a force to the blocking member to move the blocking member toward the second support portion.

12. The lifting platform according to claim 11, wherein
the elastic component is disposed on an outer peripheral side or an inner side of the blocking member.

13. The lifting platform according to claim 11, wherein the elastic component is accommodated within the first housing, one end of the elastic component abuts against the first housing, and the other end thereof abuts against the blocking member.
